(19) United States
(12) Patent Application Publication
(10) Pub. No.: US 2010/0047848 A1
(43) Pub. Date: Feb. 25, 2010

(54) Colorimetric determination of somatic cell count in milk

(76) Inventors: Wai Tak Law, Moorestown, NJ (US); Robert Douglas Harper, Marlton, NJ (US)

Correspondence Address: Donald C. Simpson, Esq., 126 Borton Landing Road, Moorestown, NJ, 08057-3055, US

(21) Appl. No.: 12/589,626

(22) Filed: Oct. 26, 2009

Related U.S. Application Data
(63) Continuation-in-part of application No. 11/512,498, filed on Aug. 30, 2006, now abandoned.

(51) Int. Cl. C12Q 1/02; C12M 1/34

(52) U.S. Cl. 435/29; 435/288.7

(57) ABSTRACT

A simple calorimetric in-line quantitative test to measure white blood cell counts in milk samples using a liquid reagent system that simplifies quantitative in-line SCC measurements using a reflectance measuring mode, and a new apparatus, which permits in-line colorimetric analysis.

### CROSS REFERENCE TO RELATED APPLICATIONS

This application is a continuation-in-part of U.S. patent application Ser. No. 11/512,498 filed Aug. 30, 2006, the priority of which is hereby claimed.

### FEDERALLY SPONSORED RESEARCH AND/OR DEVELOPMENT

The present invention was partially supported by a grant from USDA, grant no. 2007-33610-18447, and the U.S. government has some rights in this invention.

### BACKGROUND OF THE INVENTION

Mastitis is an inflammation of the mammary gland in an animal's udder that costs the dairy industry great economic loss. The dairyman generally is aware of clinical mastitis because a swollen udder can be observed, or the milk is watery, thick or ropy. Unfortunately, an apparently healthy animal can harbor sub-clinical mastitis, which makes up about 70% of the mastitis in dairy herds. Infections may continue for weeks before abnormal milk or soreness of the udder is observed. Mastitis in dairy herds is a major contributor to decreased milk quality and many believe that mastitis is a food safety and animal welfare issue.

Current practice for controlling mastitis is to monitor the Somatic Cell Counts (SCC) of milk samples from bulk tanks or from individual cows. Samples are collected and sent to laboratories for quantitative assays using specialized instruments such as flow cytometers. The instruments used are usually large and costly, and requiring trained personnel to operate. The turn around time for these assays is usually days.

SCC in milk has become the universal means of screening and monitoring mastitis. Bulk tank milk somatic cell counts (BTSCC) are a measure of the prevalence of mastitis in a dairy herd, and are used by regulatory agencies as an indicator of the wholesomeness, safety and suitability of raw milk for human consumption. The upper limit for BTSCC establishes the amount of abnormal milk tolerated in the supply. The European Union, New Zealand, Australia, Switzerland, Norway and Canada all accept 400,000 cells/mL as the upper limit, while the United States is 750,000 cells/mL. SCC is commonly measured off-line in laboratories. The traditional, available cow-side test is the California Mastitis Test (CMT). The CMT reagent is a detergent with a color indicator added. When milk and the reagent are mixed in equal amounts, the reagent dissolves or disrupts the outer cell wall and the nuclear cell wall of any white blood cell (WBC), releasing DNA that gels to form a stringy mass. As the number of WBC increase, the amount of gel formation will also increase. The gel formation is then scored or read for possible infection. The CMT reagent is inexpensive, but the test results are highly user-dependent, and the sensitivity of the method is low, while the false positive rate is sometimes as high as 50%.

Electrical conductivity methods, such as the MAS-D-TEC® device, are an electrode based system that can measure conductivity of milk sample at the cow-side. The principle of this test is based on the observation that milk electrolytes such as sodium and chloride increase when SCC is high. The test is simple to use, but has the drawback of low sensitivity and requires individual calibration for each cow.

New generations of cow-side testing have also been commercialized. They are represented by the nine pound DeLaval cell counter (DCC) that uses a disposable test cassette to estimate cell counts by digital imaging (U.S. Pat. No. 6,919,960), and the PortaSCC® milk test and reader (PortaCheck) that estimates WBC counts by an enzymatic reaction (U.S. Pat. No. 6,709,868). These analyzers have enabled users to obtained quantitative SCC data quickly at the cow-side, and are useful tools for the management of mastitis. However, these cow-side tests still require manual labor to run.

Many attempts have been made to bring faster testing to the cow-side. The recent introduction of automatic milking systems (AMS) have the potential to enhance quality of life for dairy producers and their cows, as well as increase milk production and milk quality. Dairy farm sizes are also increasing with time, with the increasing need for better management of the cows. There has been increasing interest in the development of new in-line sensors. In-line SCC sensors are designed to take samples directly from the milking lines and measure signals that may reflect the health of an animal. This approach will be ideal for evaluating up-to-date data for each animal in real time. The measurements of milk color and conductivity are the two most popular methods being adapted to in-line measurements. The color sensor measures the presence of the red color of blood. The presence of blood usually indicates symptoms of clinical mastitis. As infection occurs, salts and ions also come out of the inflamed, damaged tissues and leak into the milk. In solution, ions enable the flow of electricity, so the more leaked ions, the greater the conductivity. Consequently, changes in conductivity can be indicators of SCC. Robar (U.S. Pat. No. 3,989,009) taught about the use of conductivity measurements to estimate bovine mastitis. The use of conductivity sensors has been thoroughly investigated and results are not satisfactory. Not all mastitis cases show increases in electrical conductivity of milk and in addition, many increases in conductivity may not be due to mastitis, resulting in a great number of false positives. In most cases, instruments based on color or conductance can only alert the dairymen the presence of clinical mastitis. Sensortec in New Zealand has developed an in-line Somatic Cell Count Sensor based on CMT technology. They have automated and standardized the viscosity measurement of DNA-gel formation. The rate of flow of gel formed from a mixing chamber into waste chamber is proportional to DNA, which is proportional to SCC. There are several disadvantages to this system-these include gel clogging of orifices, milk reological differences due to protein and fat content, and length of assay. This method also requires a rather specialized instrument and produces only semi-quantitative SCC measurements in 5 ranges.

Other technologies for in-line SCC measurements have also been reported. Hansen (U.S. Pat. Nos. 6,731,100, 6,919,960) described a method that labels the cells with stain and estimates cell counts by a detection element such as CCD array. This method is similar to the flow counting method but not suitable for in-line SCC measurements. Tesnkova (U.S. Pat. No. 6,793,624) presented a method of using irradiating light in a wavelength range of 400-2,500 nm, together with multivariant analysis to diagnose the presence of mastitis in cows. The method would have been an ideal non-contact sensor. However, this method was found to be highly affected by interfering substances. Mangan (U.S. Pat. No. 6,307,362) described an in-line SCC analyzer using sodium ion measurement. Like conductivity measurements, the correlation to SCC was low. Both Tassitano (U.S. Pat. No. 5,628,964) and Bullock (U.S. Pat. No. 4,376,053) taught the use of an in-line filter or release mechanism to detect clot formation These methods are only suitable for picking up milk samples that exhibit severe clinical mastitis symptoms.

There remains a need for a simple, in-line, accurate cow-side test for the quantitative determination of SCC.

### SUMMARY OF THE INVENTION

The present invention involves using a simple calorimetric method for the in-line quantitative test to measure white blood cell counts in milk samples. The invention uses a liquid reagent based system that makes it easy for quantitative in-line SCC measurements that was not possible with prior arts. The invention includes a new analytical method that uses a reflectance measuring mode, and a new apparatus, which permits in-line calorimetric analysis.

### BRIEF DESCRIPTION OF THE DRAWINGS

FIG. 1 is a schematic of an embodiment of the in-line apparatus of the present invention.

FIG. 2 is a graphical representation of the data of Example 1 using a Minolta reflectometer.

FIG. 3 is a schematic enlargement of a signal portion for the embodiment of FIG. 1 in which the light reflectance signal system has sensors 32 placed at 90-degree angle to the flow cell surface.

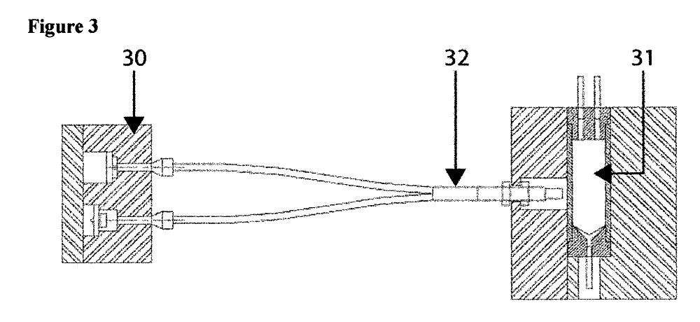

FIG. 4 is a schematic enlargement of a signal portion for the embodiment of FIG. 1 in which the light reflectance signal system has sensors 52 placed at 45-degree angle to the flow cell surface.

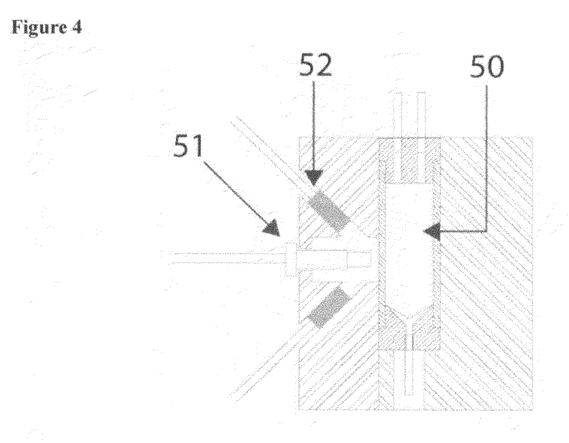

FIG. 5 is a graphical representation of the data obtained in Example 2 using the optical system shown in FIG. 4.

### DETAILED DESCRIPTION OF THE INVENTION

Since over 90% of somatic cells are WBC or leukocytes, the proposed method will directly determine the somatic cell count, yielding quantitative results of individual milk at the cow-side. The proposed analytical system will use an inexpensive photometer and liquid reagents for detection, and will produce accurate quantitative SCC measurements in approximately one to two minutes per assay.

All somatic cells or leukocytes have an enzyme called esterase on their cell wall. The role of the polymorphonuclear leucocytes esterase is to convert acetates to phenols. Over the years, urine test-strips have been used to detect the presence infection by indicating the presence of leukocytes in the urine. However, due to the interferences in sample matrixes such as blood and milk, no field test for leukocytes was available until PortaScience published a new technology in 2004. The novel SCC milk test was based on a solid phase test format, and a new dye substrate, 3-(N-tosyl-L-alanyloxy)-indol (Taloxin) (U.S. Pat. No. 6,709,868), which is very sensitive to esterase, yielding a strong blue color in the presence of esterase. The enzyme catalyses the hydrolysis of dye-substrate, and forms an indigo blue colored dye as the reaction product.

The concentration of leukocytes and WBC in milk (SCC) is proportional to the enzyme esterase presence, which is proportional to the end color intensity of the indigo dye. This enzymatic reaction has been commercialized successfully for semi-quantitative measurement of leukocytes in urine (U.S. Pat. No. 4,278,763), and recently a quantitative solid phase cow-side test-the PortaSCC milk test-has also been commercialized (U.S. Pat. No. 6,709,868). Potentially this method is an excellent candidate for the development of an in-line SCC test. However, because of the insolubility of the dye substrate in water and the interferences in the milk sample, no liquid reagent using this principle was ever reported for an in-line application. It was surprising, therefore, to find that we have identified a dual reagent system that keep the dye substrate soluble, accelerates the reaction, and reduced interferences, allowing for a rapid detection of SCC (<90 seconds) in liquid phase. Since milk samples are opaque, it is not easy to monitor the color change in the reaction mixture using traditional light transmittance method. We also found that a simple LED/silicon detector optical system was able to measure the reflectance of the resulting color changes quantitatively, allowing for the first time a simple and inexpensive in-line SCC measurement system to be constructed.

The active reagents of the invention consist of a dye substrate component and a separate buffer component. It was found to be critcal that the buffer reagent be added to the milk sample and dispersed therein prior to the addition of the dye substrate. The preferred dye substrate used in the reagent system is a member of the indoxyl ester family, such as 3-acetyl indoxyl and 3-(N-tosyl-L-analyloxy)-indole dissolved in low molecular alcohols. However, any know substrate that can be hydrolyzed by the esterase on white blood cells to form a colored dye can be used. The buffer component works best at a pH of greater than 9.0, but can be functional between pH 7.0-11.0 and at concentrations between 0.01M to 2M. A representative and preferred buffer is tris(hydroxymethyl) aminomethane, commonly referred to as “Tris”. A surfactant such as the non-ionic surfactant Triton×100 in the buffer helps to disperse the cell components in the assay mixture, and many other non-ionic, anionic, or cationic surfactants are suitable for this purpose.

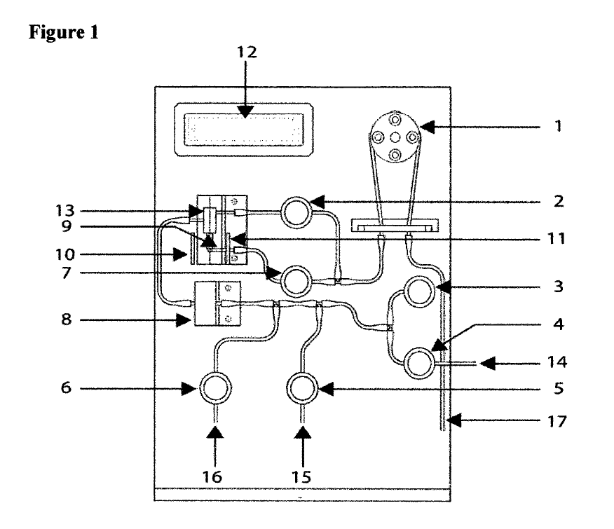

The in-line analyzer of the invention consists of a fluid control system, an optical detection system, and related electronics and display, see FIG. 1. Optionally, a temperature control system can be added to the system.

### Example 1

### Liquid Reagents for SCC Determination

The reagent components of the invention consist of the following formulation:

Reagent 1: Taxolin, 10 mg/mL of isopropanol

Reagent 2: Tris buffer, 1 molar, pH 9.8 at 24° C.; Triton X-100, 15 mg/mL of buffer

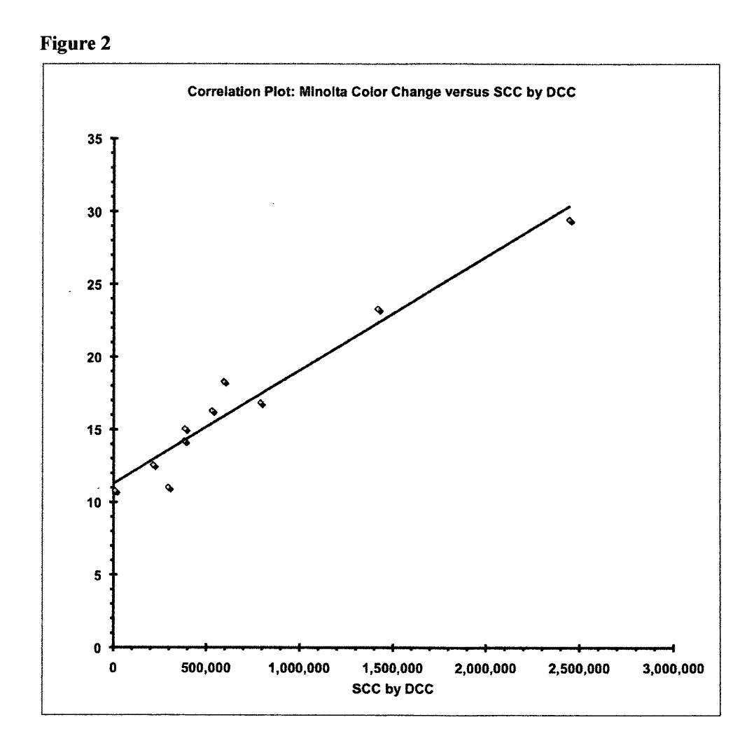

Ten fresh milk samples were collected for this study. One hundred microliters of the reagent is mixed with 100 μL of fresh milk sample, and the color changes measured by a Minolta CR-321 calorimeter in Hunter's units in 180 seconds were plotted against the Deleval's Direct cell counter (DCC) method. The data is summarized in Table 1, and the correlation shown in FIG. 2.

TABLE 1
Correlation of the Present In-Line method versus DCC

| | | Minolta |
|---|---|---|
| | | Color |
| Sample | SCC by DCC | Change |
| 1 | 7,000 | 10.8 |
| 2 | 214,000 | 12.57 |
| 3 | 382,000 | 14.22 |
| 4 | 530,000 | 16.3 |
| 5 | 1,417,000 | 23.3 |
| 6 | 385,000 | 15.05 |
| 7 | 790,000 | 16.85 |
| 8 | 2,445,000 | 29.45 |
| 9 | 593000 | 18.31 |
| 10 | 295,000 | 11.03 |

### Example 2

### In-Line SCC Determination

The milk sample from the milking line is introduced to the in-line instrument flow cell by a pump or pumps and a series of valves, where it is mixed with the reagents. After a fixed incubation period, the color intensity is read in a reflectance mode.

The schematic of the in-line instrument is shown in FIG. 1.
1. Fluidic controls—The instrument design has one peristaltic pump [FIG. 1:1], and six valves controlling sample and reagents measurements [FIG. 1:2-7], mixing, and washing steps required in the assay protocol. The peristaltic pump was selected over direct drive pump because of the proven reliability and low cost. However, a step counter was added to ensure accurate measurements of liquid volumes. The number of valves can be reduced to three, but using six valves will simplify the design of the sequencing. Optionally, three or more pumps can also be used for the system to simplify the fluidic system. The instrument also contains a reagent bottle, a buffer bottle, and a waste bottle.
2. Optical detection—An optical flow cell [FIG. 1:9] with a path length of 1-10 mm, an emitter board that uses light emitting diodes (LED) as light source [FIG. 1:10], and a sensor [FIG. 1:11], for example, a silicon detector, is used to measure the optical intensity of the color of the reaction mixture. A liquid crystal display (LCD) [FIG. 1:12] displays the SCC as a digital read out. Electronic control boards are used to control the fluid movements and the signal processing. The optical signal change is measured by reflected light. The light source is directed to the flow cell surface by an optical fiber, and the reflectance measurement was guided back to the sensor using another optical fiber. The angle of reflectance measurement can range from 1-90 degrees from the flow cell surface. The light intensity reflected from the surface of the milk and reagent mixture inside the flow cell is measured.
3. Optional Temperature control—A temperature controlled heating element will be designed into the back of the flow cell. The purpose will be to keep the assay temperature constant at 37°-40° Celsius. Since the principle of the reagent is enzymatic based, keeping a constant reaction temperature will ensure the accuracy of the test. A side benefit of running the reaction at slightly elevated temperature is the increase in the reaction rate, which in turn will help decrease the assay time.

The in-line assay protocol using a flow cell can be used with many different combinations of reagents and milk sample mixture ratio. Assay times can also be optimized according to the reagent formulations; one example is summarized as follows:
(1) 100 μL sample of milk is introduced into the flow cell.
(2) 100 μL aliquot of buffer reagent is introduced.
(3) Followed by 60 μL of dye substrate reagent.
(4) The solution is mixed for 5 seconds in the flow cell.
(5) The reflectance reading of the colored mixture is read at 90 seconds. Color intensity is proportional SCC count.
(6) A 500 μL aliquot of buffer washes the mixture in the flow cell into waste.
(7) Steps 1 through 6 are repeated with a 100-180 second turnaround for each milk sample.

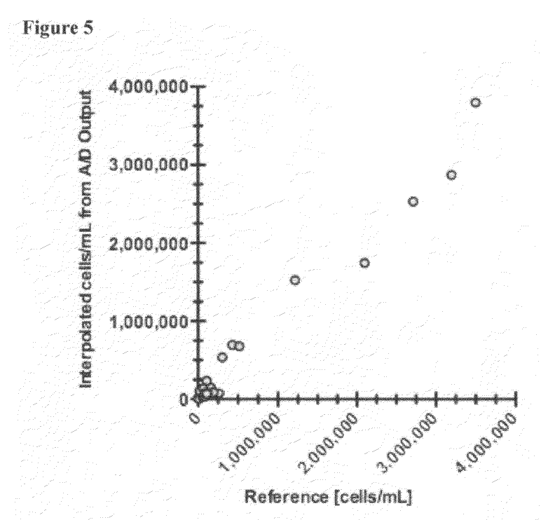

Thirty fresh milk samples were assayed with a reference SCC method (FOSS) and the calibrated in-line instrument with 45-degree placement angles of the sensors. The correlation chart and result table are shown in FIG. 5. With the range of 2,000 to 3.5 million cells/mL, the correlation coefficient (r) was 0.989. The data suggests that the in-line instrument can produce SCC data with acceptable accuracy when compared to commercial reference laboratory grade instruments.

1. A method for the determination of the somatic cell count in milk comprising mixing a milk sample to be analyzed with a dual reagent system for colorimetric measurements. The first reagent comprising a dye substrate dissolved in a low molecular weight alcohol. The second reagent is a buffer solution in concentrations between 0.01M to 2M and adapted to maintain the system at a pH in the range of 7.0 to 11.0, and measuring the colorimetric change in the milk mixture based on an enzymatic reaction using a reflectance mode.

2. An apparatus for the in-line calorimetric determination of somatic cell count in milk comprising in combination one or more peristaltic pumps and valves, a flow cell, a supply of dye substrate reagent, a supply of buffer reagent, means for delivering the dye substrate and said buffer to said flow cell, a separate means for delivering a milk sample for analysis to said flow cell, after said substrate and said buffer have been mixed, and means for measuring calorimetric change of the reagent milk mixture by reflected light.
